(19) United States
(12) Patent Application Publication
(10) Pub. No.: US 2015/0142008 A1
(43) Pub. Date: May 21, 2015

(54) CLAMPING STRUCTURE FOR UMBILICAL CORD

(71) Applicant: HUI LING HSIAO, CHANGHUA COUNTY (TW)

(72) Inventor: HUI LING HSIAO, CHANGHUA COUNTY (TW)

(21) Appl. No.: 14/513,378

(22) Filed: Oct. 14, 2014

(30) Foreign Application Priority Data
Nov. 15, 2013 (TW) 102221397

(51) Int. Cl. A61B 17/122

(52) U.S. Cl. CPC A61B 17/122
USPC 606/120

(57) ABSTRACT

A clamping structure for umbilical cord includes a body formed as bar shaped. The lateral surface of the body includes a rack section formed by engaging teeth distantly and straightly aligned. The rack section further includes sharp teeth protruded from the lateral surface. A head portion is assembled to one of two ends of the body and a buckling hole is defined thereon. An engaging structure is assembled in the buckling hole, and a recessed rail is disposed at the engaging structure. When the other end of the body is inserted into the buckling hole, the body forms a ring with the lateral surface defined as an inner wall thereof, the engaging teeth are engaged with the engaging structure, and each sharp teeth is extended toward an interior of the ring to pass through the recessed rail so as to fasten an umbilical cord securely.

### BACKGROUND OF THE INVENTION

1. Field of the Invention

The present invention relates to a clamping structure for umbilical cord, and more particularly to clamping structure for umbilical cord with spanner band appearance.

2. Description of the Prior Art

When a newborn infant is delivered from the mother, the umbilical cord must be cut. Practically, as shown in FIG. 5, a conventional umbilical cord clamp 5 is applied for clamping two sides of the umbilical cord 6, so that the conventional umbilical cord clamp 5 abuts against the umbilical cord 6 as much as possible to close the passageway within the umbilical cord 6. However, since the infant is just delivered from the mother, amniotic fluid or other viscous fluids are still coated on the infant skin, so that the umbilical cord 6 is wet and slippery thus hard to be positioned by the conventional umbilical cord clamp 5. On the other hand, since the umbilical cord 6 is wet and slippery, the umbilical cord 6 is commonly clamped by the conventional umbilical cord clamp 5 with the position of the umbilical cord 6 distant from a pivot end 51 of the conventional umbilical cord clamp 5, so that parts of the umbilical cord 6 is exposed from the conventional umbilical cord clamp 5 and thus cannot be clamped; hence, the passageway within the umbilical cord 6 cannot be closed completely, resulting in inconvenience for the user.

The present invention is, therefore, arisen to obviate or at least mitigate the above mentioned disadvantages.

### SUMMARY OF THE INVENTION

An object of the present invention is to provide a clamping structure for umbilical cord with spanner band appearance. When the clamping structure for umbilical cord is enclosed the periphery of the umbilical cord and bind the umbilical cord securely, the passageway within the umbilical cord can be closed sufficiently, thereby improving the convenience in cutting the umbilical cord.

To achieve the above and other objects, a clamping structure for umbilical cord is provided and includes a body and a head portion. The body is formed as elongate-bar shaped and has a lateral surface. The lateral surface includes a rack section formed by a plurality of engaging teeth, in which the engaging teeth are distantly and straightly aligned on the body to form the rack section. The rack section further includes a plurality of sharp teeth protruded from the lateral surface and disposed along the lateral surface. The head portion is assembled to one of two ends of the body. A buckling hole is defined at the head portion, and an engaging structure is assembled in the buckling hole. A recessed rail is disposed at the engaging structure. Based on this, when the other end of the body is inserted into the buckling hole, the body is formed as a ring with the lateral surface defined as an inner wall of the ring, the engaging teeth of the rack section are engaged with the engaging structure, and each of the sharp teeth is extended toward an interior of the ring so as to pass through the recessed rail, thereby allowing to fasten an umbilical cord securely.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a perspective view of the present invention;

FIG. 2 is a partial enlarged view showing the rack section of the present invention;

FIG. 3 is a partial enlarged view showing the head portion of the present invention;

FIG. 4 is an operational view showing the present invention is applied to an umbilical cord; and

FIG. 5 is an operational view showing a conventional umbilical cord is applied to an umbilical cord.

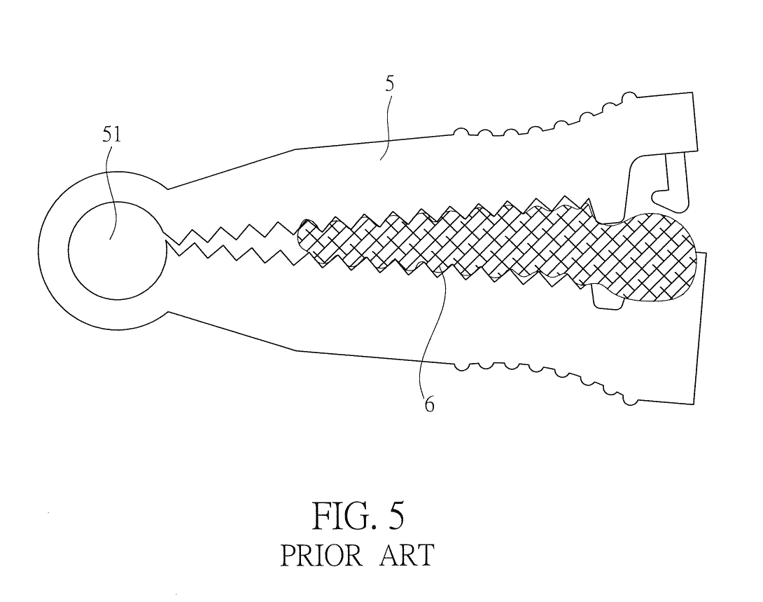

### DETAILED DESCRIPTION OF THE PREFERRED EMBODIMENTS

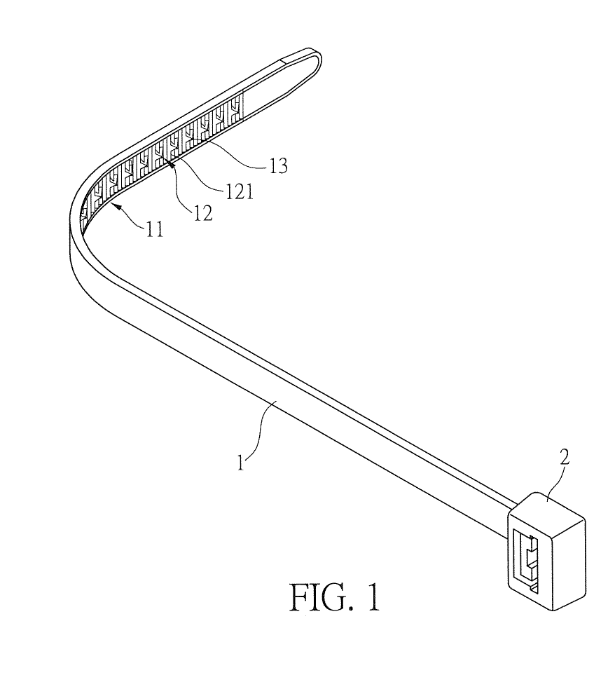

It is understood that, the structures shown in FIGS. 1-4 are exemplary embodiments of the present invention and are only provided for illustrative purposes, the scope of the present invention is not limited by the structures provided in the figures.

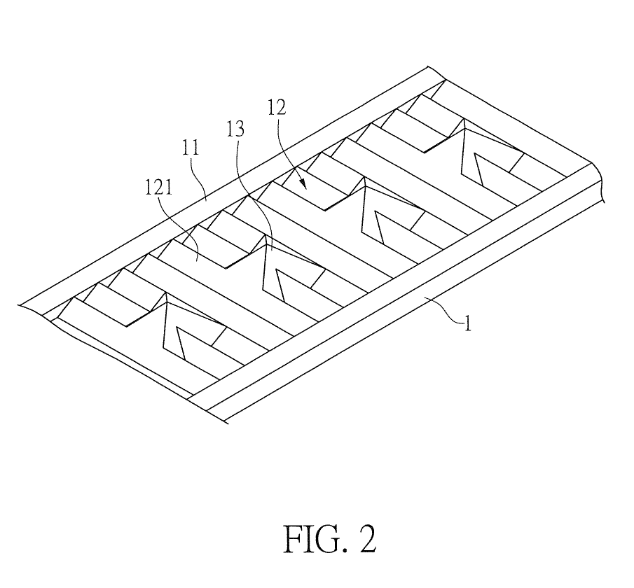

The present invention provided a clamping structure for umbilical cord. As shown in FIG. 1, the clamping structure for umbilical cord includes a body 1 formed as elongate-bar shaped. The body 1 has a lateral surface 11. The lateral surface 11 includes a rack section 12 formed by a plurality of engaging teeth 121. As shown in FIG. 2, the engaging teeth 121 are distantly and straightly aligned on the body 1 to form the rack section 12. The rack section 12 includes a plurality of sharp teeth 13 protruded from the lateral surface 11 and disposed along the lateral surface 11. In this embodiment, each of the sharp teeth 13 is disposed on the rack section 12 and goes across on tops of plural engaging teeth 121. The sharp teeth 13 are distantly aligned in the length direction of the rack section 12.

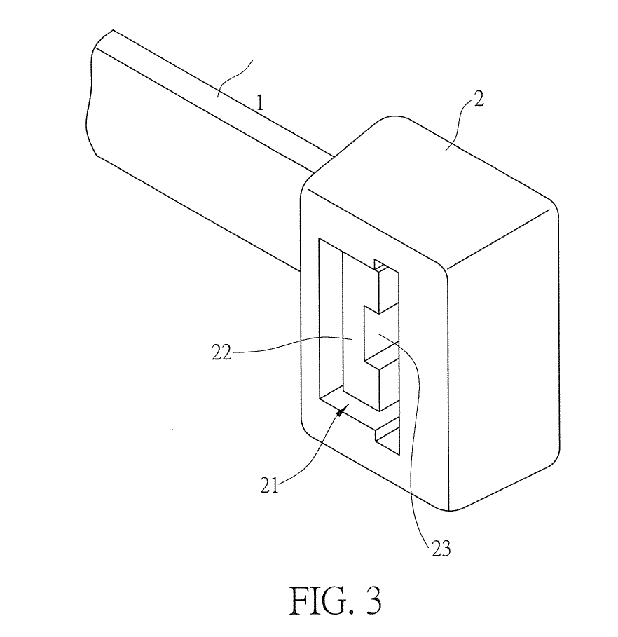

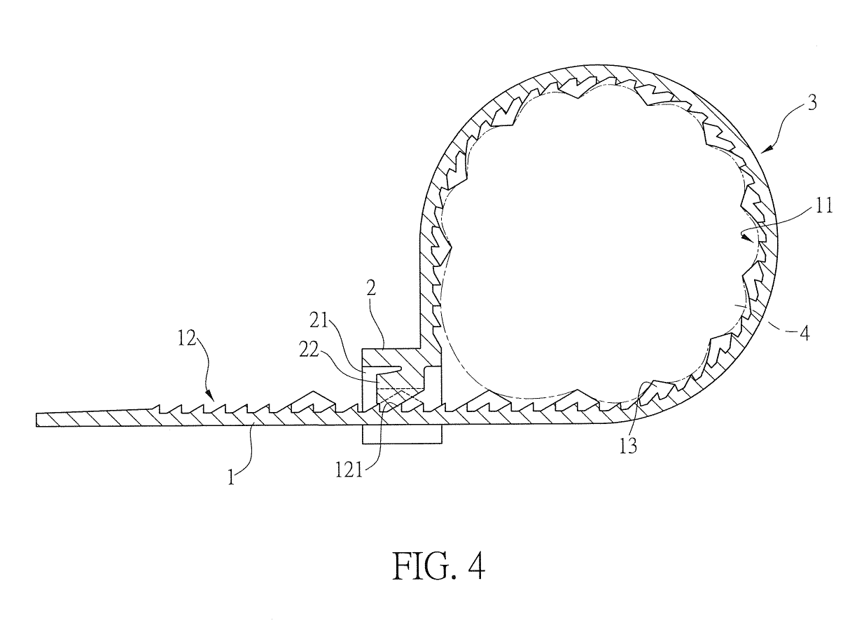

A head portion 22 is assembled to one of two ends of the body 1, as shown in FIG. 3. A buckling hole 21 is defined at the head portion 2. An engaging structure 22 is assembled in the buckling hole 21 so as to engage with the rack section 12 of the body 1. In detail, the body 1 is curled, as illustrated in FIG. 4, and the other end of the body 1 is inserted into the buckling hole 21 of the head portion 2, thereby the body 1 forming a ring 3 with the lateral surface 11 defined as an inner wall of the ring 3. Based on this, the engaging teeth 121 of the rack section 12 are engaged with the engaging structure 22 in the buckling hole 21 to confine the shape of the ring 3. The size of the ring 3 is depended on the length of parts of the body 1 passing through the buckling hole 21; that is, if the aforementioned length is longer, the size of the ring 3 will be smaller. As shown in FIG. 3 and FIG. 4, a recessed rail 23 is disposed at the engaging structure 22, thus when the body 1 is inserted into the buckling hole 21, the sharp teeth 13 can pass through the recessed rail 23.

Accordingly, the clamping structure of umbilical cord according to the present invention works well in clamping umbilical cord 4. As shown in FIG. 4, when the clamping structure of umbilical cord according to the present invention is applied to fasten an umbilical cord 4, firstly the body 1 is curled to surround an outer periphery of the umbilical cord 4, and then the body 1 is passing through the buckling hole 21 of the head portion 2, so that the engaging structure 22 is engaged with the rack section 12 so as to confine the shape of the body 1 in a ring shape. After the body 1 is passing through the buckling hole 21, the other end of the body 1 is pulled so as to reduce the size of the ring 3 thereby allowing the umbilical cord 4 be fastened by the clamping structure of umbilical cord securely. Furthermore, since the sharp teeth 13 are extended toward the interior of the ring 3 formed by the body 1, the sharp teeth 13 can securely fix on the periphery of the umbilical cord 4 even when the umbilical cord 4 is wet and slippery, thereby significantly improving the convenience in cutting the umbilical cord 4.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A clamping structure for umbilical cord, comprising:
a body, formed as elongate-bar shaped, the body having a lateral surface, the lateral surface comprising a rack section formed by a plurality of engaging teeth, the engaging teeth being distantly and straightly aligned on the body to form the rack section, and the rack section further comprising a plurality of sharp teeth protruded from the lateral surface and disposed along the lateral surface; and
a head portion, assembled to one of two ends of the body, a buckling hole being defined at the head portion, an engaging structure being assembled in the buckling hole, and a recessed rail being disposed at the engaging structure;
whereby, when the other end of the body is inserted into the buckling hole, the body is formed as a ring with the lateral surface defined as an inner wall of the ring, the engaging teeth of the rack section are engaged with the engaging structure, and each of the sharp teeth is extended toward an interior of the ring so as to pass through the recessed rail, thereby allowing to fasten an umbilical cord securely.
